(19) United States
(12) Patent Application Publication
(10) Pub. No.: US 2025/0387375 A1
(43) Pub. Date: Dec. 25, 2025

(54) SOFALCONE DERIVATIVES AND MEDICAL USE THEREOF

(71) Applicant: KAOHSIUNG MEDICAL UNIVERSITY, Kaohsiung City (TW)

(72) Inventors: Chin-Chung Wu, Kaohsiung City (TW); Chih-Hua Tseng, Kaohsiung City (TW); Po-Hung Wu, Kaohsiung City (TW); Chun-Hsiang Huang, Kaohsiung City (TW); Meng-Chun Tsai, Kaohsiung City (TW); Yu-Chi Tung, Kaohsiung City (TW)

(73) Assignee: KAOHSIUNG MEDICAL UNIVERSITY, Kaohsiung City, TW (TW)

(21) Appl. No.: 18/813,023

(22) Filed: Aug. 23, 2024

Related U.S. Application Data
(60) Provisional application No. 63/661,592, filed on Jun. 19, 2024.

(51) Int. Cl. A61K 31/426; A61K 31/122; A61K 31/341; A61K 31/381; A61K 31/44; A61P 7/02; C07C 15/12; C07D 211/04; C07D 307/36; C07D 327/04; C07D 333/10

(52) U.S. Cl. CPC A61K 31/426; A61K 31/122; A61K 31/341; A61K 31/381; A61K 31/44; A61P 7/02; C07C 15/12; C07D 211/04; C07D 307/36; C07D 327/04; C07D 333/10

(57) ABSTRACT

A series of sofalcone derivatives having the effect of inhibiting platelet aggregation and antithrombotic effect are disclosed. These sofalcone derivatives are antagonists of TxA2 receptor and have the effect of inhibiting platelet activation and aggregation without causing bleeding. In addition, a preparing method of the sofalcone derivatives and a method for preventing or treating thrombotic diseases or inhibiting platelet aggregation by administrating the sofalcone derivatives are also disclosed.

### CROSS-REFERENCE TO RELATED APPLICATIONS

This application claims the benefit of U.S. Provisional Patent Application No. 63/661,592, filed on Jun. 19, 2024, which is incorporated by reference herein in its entirety.

### FIELD OF THE INVENTION

The present invention relates to a series of sofalcone derivatives, which have effects of inhibiting platelet aggregation and antithrombotic effects. The present invention also relates to a method for using the sofalcone derivatives to treat thrombotic disease or inhibit platelet aggregation.

### DESCRIPTION OF PRIOR ARTS

Thromboxane A2 (TxA2) is the main metabolite of arachidonic acid (AA) in platelets. When the platelets are activated, AA is released from the phospholipids in the cell membrane to the cytoplasm, where it is converted into prostaglandin (PG) G2/H2 by cyclooxygenase (COX), and then converted to TxA2 by TxA2 synthase. TxA2 can be released out of the platelet cells and act on the thromboxane A2 receptor on the platelet membrane, causing platelet activation and aggregation. Therefore, TxA2 is an important enhancement mechanism for platelet activation. Aspirin reduces platelet TxA2 synthesis by inhibiting COX, thereby producing antiplatelet effects. However, Aspirin can also inhibit prostaglandin synthesis in other cells, for example, inhibiting prostaglandin I2 (PGI2) synthesis by vascular endothelial cells and inhibiting prostaglandin E2 (PGE2) synthesis by gastric cells. PGI2 inhibition may counteract the antiplatelet effect of Aspirin, while PGE2 inhibition may be related to the gastrointestinal ulcer caused by Aspirin. Therefore, the development of drugs that can antagonize TxA2 receptors or selectively inhibit TxA2 synthase may be an antiplatelet therapy strategy superior to Aspirin.

There are two types of TxA2 receptor antagonists currently available in clinical use: seratrodast and ramatroban, but they are only used for treating asthma or allergic rhinitis and have not yet been approved for antiplatelet therapy. Picotamide is a dual-effect antagonist for TxA2 synthase and TxA2 receptor, but its potency is low. As a result, currently, it is not widely used in clinical antiplatelet therapy. Therefore, the development of novel TxA2 receptor antagonists is still of urgent importance.

Sofalcone is a compound of the chalcone class, which is a derivative obtained by artificial synthesis and modification of sophoradin, a component of Sophora subprostrata. Sophora subprostrata is commonly used in traditional Chinese medicine, and its traditional uses include clearing away heat and toxin, soothing throat and reducing swelling. Recent studies have found that Sophora subprostrata has an anti-gastric ulcer effect, and it is confirmed that its active ingredient is sophoradin. Taisho Pharmaceutical Co. Ltd. in Japan developed sofalcone through artificial synthesis and chemical modification based on the chemical structure of sophoradin, and launched it onto the market in 1984 as an anti-gastric ulcer drug. Because of its safety and effectiveness, Japan further approved it as an over-the-counter drug in 1999. Previous studies showed that the mechanism of sofalcone's anti-gastric ulcer effect was related to an increased level of PGE2 in gastric tissues by sofalcone's inhibitory activity against 15-hydroxy-prostaglandin dehydrogenase (15-PGDH). In addition, two metabolites of sofalcone in human body also have anti-gastric ulcer effects. In addition to its anti-gastric ulcer effect, when being used in combination with rabeprazole, amoxicillin and clarithromycin, sofalcone can enhance the effect of inhibiting Helicobacter pylori infection of gastric tissues, thereby achieving the effect of anti-gastritis.

Recent studies also pointed out that sofalcone could not only inhibit inflammatory reactions between macrophages and adipocytes, but also reduce differentiation of preadipocytes into adipocytes, indicating that sofalcone may be used in diseases such as obesity and metabolic syndrome. However, up until now, there is no study on the effects of sofalcone on cardiovascular diseases, especially on its antiplatelet effects.

### SUMMARY OF THE INVENTION

The present invention provides a sofalcone derivative, which has the effect of inhibiting platelet aggregation and antithrombotic effect.

The present invention further provides a method for inhibiting platelet activation and aggregation in a subject without causing bleeding, which comprises administering a composition comprising a sofalcone derivative to the subject.

The present invention further provides a method for preventing or treating thrombotic disease, which comprises administering a composition comprising a sofalcone derivative to a subject suffering from thrombotic disease.

### BRIEF DESCRIPTION OF THE DRAWINGS

FIG. 1 shows methods for synthesizing sofalcone derivatives.

FIG. 2 shows methods for synthesizing sofalcone derivatives. MIl: methyl iodide.

FIG. 3 shows a method for synthesizing a sofalcone derivative.

FIG. 4 shows methods for synthesizing sofalcone derivatives. DCM: Dichloromethane, KI: Potassium iodide. DMF: Dimethyl formamide.

FIGS. 5A to 5C show that sofalcone and its derivatives can competitively antagonize U46619-induced platelet aggregation. FIG. 5A shows that sofalcone (SFC) can competitively antagonize U46619-induced platelet aggregation. FIG. 5B shows that sofalcone derivative 3 can competitively antagonize U46619-induced platelet aggregation. FIG. 5C shows that sofalcone derivative 30 can competitively antagonize U46619-induced platelet aggregation. After human platelet suspension was pretreated with dimethyl sulfoxide (DMSO) or sofalcone (SFC) (FIG. 5A), sofalcone derivative 3 (FIG. 5B), and sofalcone derivative 30 (FIG. 5C), different concentrations of U46619 (0.1-10 μM) were added to stimulate platelet aggregation reaction. Data are presented as mean±standard error of the mean (n=5).

FIGS. 6A and 6B show the effect of sofalcone and its derivatives on TxB2 and PGE2 productions induced by arachidonic acid (AA). FIG. 6A shows the effect of sofalcone (SFC) and its derivatives on arachidonic acid-induced TxB2 production. FIG. 6B shows the effect of sofalcone and its derivatives on PGE2 production induced by arachidonic acid. After human platelet suspension was pretreated with dimethyl sulfoxide (DMSO) or sofalcone (SFC), sofalcone derivative 3, and sofalcone derivative 30, arachidonic acid (100 μM) was added to stimulate the productions of arachidonic acid metabolites TxB2 and PGE2. Data are presented as mean±standard error of the mean (n>5). Compared with the control group, *P<0.05, **P<0.01, ***P<0.001. ASA: Aspirin.

FIGS. 7A and 7B show that sofalcone and its derivatives have antithrombotic effects in the artery. FIG. 7A shows that sofalcone (SFC) has antithrombotic effects in the artery. FIG. 7B shows that sofalcone derivative 17 has antithrombotic effects in the artery. Mice were orally administered by gavage with sofalcone (SFC, once daily for 4 days), derivative 17, or aspirin (ASA) (single administration only). The mice are then treated with ferric chloride solution to induce carotid artery thrombosis, and an ultrasound imaging system was used to monitor the time required for the thrombus to block the carotid artery (occlusion time). Data are presented as mean±standard deviation error of the mean (n≥6). Compared with the control group, *P<0.05, ***P<0.001.

FIGS. 8A and 8B show that sofalcone and its derivatives do not affect normal hemostatic function. FIG. 8A shows that sofalcone (SFC) does not affect normal hemostatic function. FIG. 8B shows that sofalcone derivative 17 does not affect normal hemostatic function. Mice were orally administered by gavage with sofalcone (SFC, once daily for 4 days), derivative 17, or aspirin (ASA) (single administration only). Then the mouse's tail was transected and the tail bleeding time was recorded. Data are presented as mean±standard error of the mean (n≥6). Compared with the control group, ***P<0.001. n.s.: No significant difference.

### DETAILED DESCRIPTION OF THE INVENTION

The present invention confirms that sofalcone has antiplatelet activity and its mechanism of action is as a TxA2 antagonist. The present invention further comprises modifying the chemical structure of sofalcone to synthesize a series of novel chemical derivatives, testing its anti-platelet aggregation activity, and optimizing the pharmacophore through structure-activity relationship analysis.

The sofalcone derivatives disclosed in the present invention can excellently and effectively inhibit platelet aggregation induced by the TxA2 receptor agonist U46619 or by collagen, and animal experiments also show antithrombotic effects without affecting the hemostatic function. Therefore, the sofalcone derivatives disclosed in the present invention can be used as novel platelet inhibitors that are effective and have a lower risk of bleeding side effects.

As used herein, the terms “a” or “an” are used to describe elements and components of the present invention. This terminology is used only for convenience and to provide a basic concept of the present invention. Furthermore, this description should be understood to include one or at least one and, unless the context clearly dictates otherwise, singular terms include the plural and plural terms include the singular. When used in conjunction with the word “comprising” in the claims, the term “a” or “an” may mean one or more than one.

The term “or” as used herein may mean “and/or”.

The present invention provides a compound or a pharmaceutically acceptable salt thereof, wherein the compound has a structure of formula (I):

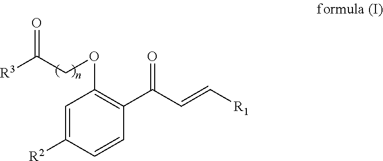

wherein R¹ is none, H, phenyl (Ph), thiophenyl, furanyl, pyridinyl, bromothiophenyl, thiazolyl, or X-substituted phenyl, and X is H, halogen, NO₂, NH₂, NHAc, O-Ac, O-geranyl, C₁₋₆ alkyl, O—C₁₋₁₀ alkyl, OH, OBn, aminothio, O-isoprenyl, O-halobenzyl, O—C₁₋₆ alkoxybenzyl, O—C₁₋₆ alkyl-CO-phenyl, or O—C₁₋₆ alkyl-COO—C₁₋₆ alkyl;
R² is none, H, OH, halogen, O-geranyl, O-isoprenyl, O—C₁₋₁₀ alkyl, O-Ac, OBn, O-halobenzyl, O—C₁₋₆ alkyloxybenzyl, phenyl-O-isoprenyl, O—C₁₋₆ alkyl-CO-phenyl, or O—C₁₋₆ alkyl-COO—C₁₋₆ alkyl;
R³ is OH or O—C₁₋₆ alkyl; and
n is 1-7.

In one embodiment, the halogen comprises fluorine (F), chlorine (C₁), bromine (Br), iodine (I) and acetonitrile (At). In a preferred embodiment, the halogen is fluorine or chlorine.

In one embodiment, the C₁₋₆ alkyl comprises C₁₋₃ alkyl.

In one embodiment, the O—C₁₋₁₀ alkyl comprises O—C₁₋₈ alkyl. In a preferred embodiment, the O—C₁₋₈ alkyl comprises the O—C₁₋₆ alkyl. In a more preferred embodiment, the O—C₁₋₆ alkyl comprises the O—C₁₋₃ alkyl.

In another embodiment, the O—C₁₋₆ alkoxybenzyl comprises O—C₁₋₃ alkoxybenzyl.

In one embodiment, the O—C₁₋₆ alkyl-CO-phenyl comprises O—C₁₋₃ alkyl-CO-phenyl.

In another embodiment, the O—C₁₋₆ alkyl-COO—C₁₋₆ alkyl comprises O—C₁₋₃ alkyl-COO—C₁₋₃ alkyl.

In one embodiment, the n is 1-5.

In one embodiment, the R¹ is phenyl-4-fluoro, the R² is H, the R³ is OH, and the n is 5.

In one embodiment, the R¹ is phenyl-4-O-isoprenyl, the R² is O-isoprenyl, the R³ is OH, and the n is 3-7. In a preferred embodiment, the R¹ is phenyl-4-O-isoprenyl, the R² is O-isoprenyl, the R³ is OH, and the n is 3.

The present invention provides a composition, which comprises a compound or a pharmaceutically acceptable salt thereof, wherein the compound has the structure of formula (I).

The present invention further provides a use of a composition for preparing an antiplatelet drug, wherein the composition comprises a compound or a pharmaceutically acceptable salt thereof, and the compound has the structure of formula (I).

The present invention further provides a use of a composition for preparing a drug for preventing or treating diseases related to platelet aggregation, wherein the composition comprises a compound or a pharmaceutically acceptable salt thereof, and the compound has the structure of formula (I).

The present invention further provides a method for preventing or treating thrombotic disease, which comprises administering a composition comprising a compound or a pharmaceutically acceptable salt thereof to a subject suffering from thrombotic disease, wherein the compound has the structure of formula (I).

The present invention also provides a compound or a pharmaceutically acceptable salt thereof, wherein the compound has a structure of formula (II):

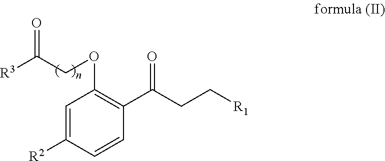

wherein R¹ is phenyl, or Y-substituted phenyl, and Y is halogen, OH, O-isoprenyl, or O—C₁₋₆ alkyl;
R² is H, OH, O-isoprenyl, or O—C₁₋₆ alkyl;
R³ is OH, O—C₁₋₆ alkyl; and
n is 1-7.

In one embodiment, the halogen is fluorine.

In another embodiment, the n is 1-5.

The present invention provides a composition which comprises a compound or a pharmaceutically acceptable salt thereof, wherein the compound has the structure of formula (II).

The present invention further provides a use of a composition for preparing an antiplatelet drug, wherein the composition comprises a compound or a pharmaceutically acceptable salt thereof, and the compound has the structure of formula (II).

The present invention further provides a use of a composition for preparing a drug for preventing or treating diseases related to platelet aggregation, wherein the composition comprises a compound or a pharmaceutically acceptable salt thereof, and the compound has the structure of formula (II).

The present invention further provides a method for preventing or treating thrombotic disease, which comprises administering a composition comprising a compound or a pharmaceutically acceptable salt thereof to a subject suffering from thrombotic disease, wherein the compound has the structure of formula (II).

The term a “pharmaceutically acceptable salt” used herein refers to derivative compounds obtained by modifying the compounds into acids or bases and salts thereof. However, there is no limitations on the types of salt as long as they are physiologically acceptable to users.

As used herein, the term “preventing” refers to suppressing or avoiding symptoms of a particular disease, disorder, symptom or side effect. As used herein, the term “treating” is meant to comprise alleviating or eliminating a disorder, disease, or one or more symptoms associated with the disorder, disease, or condition; or alleviating or eliminating the cause of the disorder, disease, or symptom itself.

As used herein, the term “subject” may be a mammal, preferably a human.

In the present invention, the compound having the structure of formula (I) or formula (II) has an anti-platelet aggregation effect. Furthermore, the compound having the structure of formula (I) or formula (II) can inhibit platelet aggregation activity. Therefore, the compound of the present invention, or derivatives or pharmaceutically acceptable salts thereof can be used to inhibit platelet aggregation and has antithrombotic activity but does not have a tendency to cause bleeding. Therefore, the present invention provides an antiplatelet drug, which comprises the compound having the structure of formula (I) or formula (II) or a pharmaceutically acceptable salt thereof.

In some aspects, the present invention provides a method for inhibiting platelet aggregation, which comprises administering a composition comprising a compound or a pharmaceutically acceptable salt thereof to a subject in need thereof, wherein the compound has the structure of formula (I) or formula (II). Furthermore, the present invention provides a method for preventing or treating a disease related to platelet aggregation, which comprises administering a composition comprising a compound or a pharmaceutically acceptable agent thereof to a subject suffering from the disease related to platelet aggregation, wherein the compound has the structure of formula (I) or formula (II).

Thrombotic diseases occur in response to injury or other signals that lead to recruitment of platelets to a site of injury. Since the compound having the structure of formula (I) or formula (II) provided by the present invention can inhibit platelet aggregation, it is suitable for preparing therapeutic or preventive agents for various diseases related to platelet aggregation or thrombotic disease. As used herein, the term “thrombotic disease” refers to a disease resulting from formation or presence of a thrombus within a blood vessel that can cause ischemia or infarction of a tissue to which the blood vessel supplies blood. In one embodiment, the disease related to platelet aggregation or thrombotic disease comprises arterial and cardiovascular thrombotic disease, venous and cardiovascular thrombotic disease, or thrombotic disease of the cardiac chamber or the peripheral circulation. In a preferred embodiment, the disease related to platelet aggregation or thrombotic disease comprises unstable angina, acute coronary syndrome, myocardial infarction, transient ischemic attack, cerebral stroke, atherosclerosis, peripheral occlusive arterial disease, venous thrombosis, thrombophlebitis, arterial embolism, kidney embolism, pulmonary embolism, or thrombotic diseases resulting from medical implants, devices, or procedures in which blood is exposed to artificial surfaces that facilitate thrombosis.

The compounds of the present invention may be administered in oral dosage forms such as tablets, capsules (each including sustained release or time-release formulations), pills, powders, granules, elixirs, tinctures, suspensions, syrups, and emulsions. The compounds of the present invention may also be administered by intravenous (bolus or infusion), intraperitoneal, subcutaneous or intramuscular injections. Therefore, the present invention uses dosage forms that are well known to those skilled in the medical arts. The compounds of the present invention may be administered independently, but are generally administered with a pharmaceutical carrier selected based on a chosen route of administration and standard pharmaceutical practices.

In addition, the compounds of the present invention may also be used to inhibit platelet aggregation in blood and blood products in vitro, for example, during storage or treatment from living organisms (for example, diagnostic or research uses).

In the present invention, the compound having the structure of formula (I) or formula (II) of the present invention can serve as an TxA2 receptor antagonists and may further have effects of inhibiting TxA2 formation. Also, the compounds having the structure of formula (I) or formula (II) can also inhibit enzyme activities of COX1 and COX2. Therefore, the compounds having the structure of formula (I) or formula (II) can block platelet activation and aggregation, thereby preventing thrombosis. In addition, the compounds having the structure of formula (I) or formula (II) do not affect normal hemostatic functions in the subject. Accordingly, the present invention provides a method for inhibiting platelet activation and aggregation in a subject without causing bleeding, which comprises administering a composition comprising a compound or a pharmaceutically acceptable salt thereof to the subject, wherein the compound has the structure of formula (I) or formula (II). The present invention further provides a method for inhibiting thrombosis in a subject without causing bleeding, which comprises administering a composition comprising a compound or a pharmaceutically acceptable salt thereof to the subject, wherein the compound has the structure of formula (I) or formula (II). In the present invention, the subject has or is at a greater risk of thrombosis than a normal subject.

In summary, the present invention designs and synthesizes a series of novel compounds based on the chemical structure of sofalcone, which has antiplatelet and antithrombotic effects; and does not affect hemostatic function in animal experiments. Therefore, the compounds of the present invention can be used as new antiplatelet drugs or used in antiplatelet therapy.

### EXAMPLES

The following examples are non-limiting and merely represent aspects and features of the present invention.

### Materials and Methods

### Preparation and Analysis of Sofalcone and its Derivatives

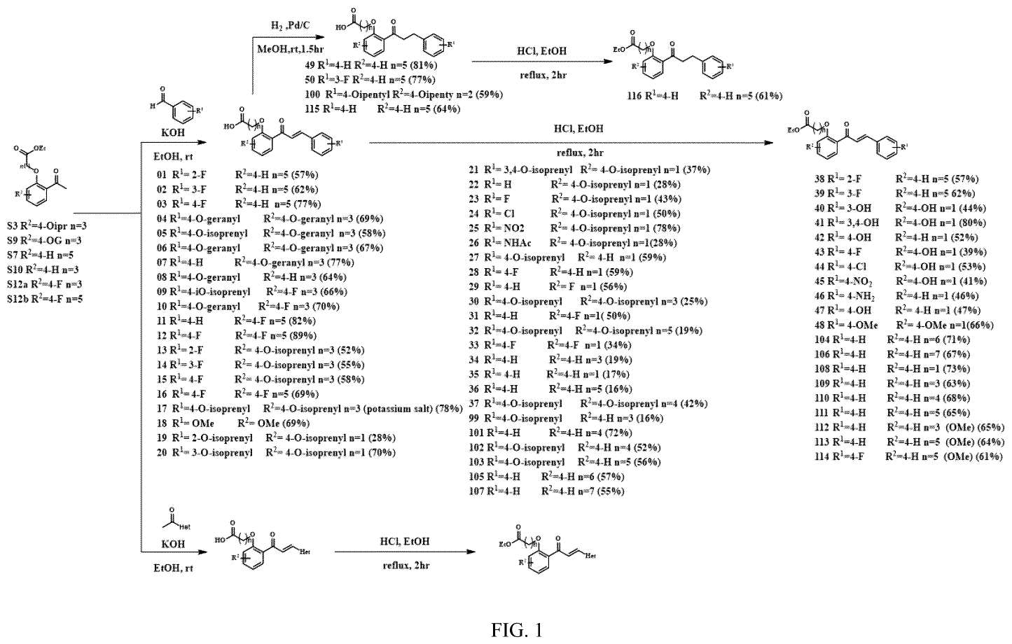

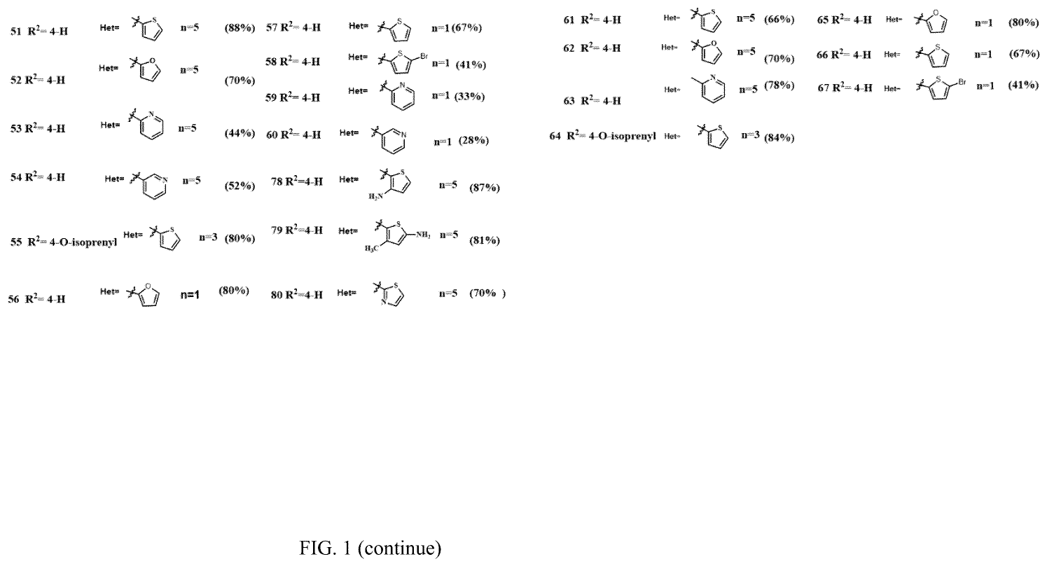

Any compound disclosed herein can be produced by methods commonly used in relevant fields or by methods taught in the examples of the present invention. The preparation methods of sofalcone and its derivatives of the present invention are shown from FIG. 1 to FIG. 4.

FIG. 1 to FIG. 4 show general synthesis methods of the sofalcone derivatives.

In the compound preparation method shown in FIG. 1, the preparation methods of Compounds 02, 39, 50, 51 and 61 are provided by the present invention as exemplar descriptions.

### Preparation of Compound 02

Starting compound S7 (0.83 g, 3 mmol) was placed in a reaction bottle, ethanol (EtOH) was the solvent, and a magnet was added to stir. 50% KOH was slowly added dropwise in and allowed it to stand for 10 minutes. Then 4-fluorobenzaldehyde (0.37 g, 3 mmol) previously dissolved in ethanol was added dropwise to the reaction bottle, and stirred at room temperature for 12-16 hours. Thin layer chromatography (TLC) was used to monitor and confirm that the reaction was complete, then precipitation of solid suspensions could be observed. The solvent was removed by using a rotary evaporator and 200 ml of water was added, acidified with 6N hydrochloric acid (HCl) until the pH value was equal to 4, and then the crude product was extracted with ethyl acetate (EA). Then purified with column chromatography, using EA:n-hexane (hex)=1:1 as the eluent. Compound 02 could be obtained by using ethanol for recrystallization.

¹H NMR (400 MHZ, CDCl₃) δ 7.67-7.72 (d, J=16 Hz, 1H, Ar—H), 7.67-7.65 (dd, J=8 Hz, 1H, Ar—H), 7.61-7.57 (m, 1H, Ar—H), 7.54 (s, 1H), 7.48-7.44 (m, 1H, Ar—H), 7.38-7.32 (m, 1H, Ar—H), 7.19-7.08 (m, 1H, Ar-H), 7.04-7.01 (m, 1H), 6.97 (s, 1H), 6.95 (s, 1H) 4.08 (m, 4H), 2.15 (m, 2H,), 1.71 (m, 2H), 1.56 (m, 2H), 1.27 (m, 2H)∘

### Preparation of Compound 39

Compound 02 (1.12 g, 3 mmol) was placed in a reaction bottle, ethanol (EtOH) was used as the solvent, and a magnet was added to stir. 3 ml of concentrated hydrochloric acid was slowly added dropwise, heated to reflux and reacted for two hours, and then monitored with a TLC sheet. After confirming completion of reaction, a rotary evaporator was used to remove the solvent, 200 mL of water was added, and the crude product was extracted with EA. Then, purified with column chromatography, and EA: n-hexane (hex)=1:1 was used as the eluent for purification to obtain Compound 39.

¹H NMR (400 MHZ, CDCl₃) δ 7.67-7.72 (d, J=16 Hz, 1H, Ar—H), 7.67-7.65 (dd, J=8 Hz, 1H, Ar—H), 7.61-7.57 (m, 1H, Ar—H), 7.54 (s, 1H), 7.48-7.42 (m, 1H, Ar—H), 7.35-7.32 (m, 1H, Ar—H), 7.19-7.06 (m, 1H, Ar-H), 7.04-7.01 (m, 1H), 6.97 (s, 1H), 6.95 (s, 1H) 4.08 (m, 4H), 2.13 (m, 2H,), 1.79 (m, 2H), 1.54 (m, 2H), 1.26 (m, 2H), 1.22 (m, 3H)∘

### Preparation of Compound 50

Compound 02 (1.12 g, 3 mmol) was placed in a reaction bottle and a palladium on carbon Pd/C (0.11 g, 1 mmol) was added, the reaction bottle was evacuated, methanol (MeOH) was used as a solvent, then hydrogen gas was introduced and reacted at room temperature for 1-3 hours. After monitoring with a TLC sheet to confirm completion of the reaction, the reaction mixture was filtered through a filter filled with diatomaceous earth under reduced pressure and the solvent was removed by using a rotary evaporator. Then, purified with column chromatography, and EA:n-hexane (hex)=1:5 was used as the eluent for purification to obtain Compound 50.

¹H NMR (400 MHZ, CDCl₃) δ 7.73 (d, J=16 Hz, 1H, Ar—H), 7.64 (dd, J=8 Hz, 1H, Ar—H), 7.61-7.56 (m, 1H, Ar—H), 7.52 (s, 1H), 7.48-7.43 (m, 1H, Ar—H), 7.37-7.32 (m, 1H, Ar—H), 7.18-6.95 (m, 3H, Ar—H), 4.05 (m, 4H), 2.19 (m, 2H,), 1.81 (m, 2H), 1.56 (m, 2H), 1.46 (m, 2H)∘

### Preparation of Compound 51

Starting compound S7 (0.83 g, 3 mmol) was placed in a reaction bottle, ethanol was used as the solvent, and a magnet was added to stir. 50% potassium hydroxide (KOH) was slowly added dropwise and allowed it to stand for 10 minutes. Then thiophene-2-carboxaldehyde (0.34 g, 3 mmol) previously dissolved in ethanol was added dropwise into the reaction bottle, stirred at room temperature for 12-16 hours. Monitored with a TLC sheet to confirm that the reaction was complete, then precipitation of solid suspensions could be observed. A rotary evaporator was used to remove the solvent, 200 ml of water was added, acidified with 6N HCl until the pH value was equal to 4, and then the crude product was extracted with ethyl acetate (EA). Then, purified with column chromatography, EA:n-hexane (hex)=1:1 was used as the eluent. Compound 51 could be obtained by using ethanol for recrystallization.

¹H NMR (400 MHZ, CDCl₃) δ 7.78-7.75 (d, J=12 Hz, 1H, Ar—H), 7.67-7.63 (dd, J=16 Hz, 1H, Ar—H), 7.46-7.42 (m, 1H, Ar—H), 7.30 (s, 1H), 7.26-7.28 (d, 1H, Ar—H), 7.07-7.04 (m, 1H, Ar—H), 7.02 (t, 1H, Ar—H), 6.96-6.74 (d, J=8,1H, Ar—H), 4.056 (m, 2H), 1.81-1.82 (m, 2H), 1.63-1.61 (m, 2H), 1.53-1.51 (m, 2H)∘

### Preparation of Compound 61

Compound 51 (1.12 g, 3 mmol) was placed in a reaction bottle, ethanol was used as the solvent, and a magnet was added for stirring. Slowly added 3 ml of concentrated hydrochloric acid dropwise, and heated to reflux for two hours. After monitoring with a TLC sheet to confirm completion of the reaction, the solvent was removed with a rotary evaporator, 200 mL of water was added and the crude product was extracted with EA. Then, purified with column chromatography, and Compound 61 could be obtained by using EA:n-hexane (hex)=1:1 as the eluent for purification.

¹H NMR (400 MHZ, CDCl₃) δ 7.78-7.75 (d, J=16 Hz, 1H, Ar—H), 7.67-7.65 (dd, J=8 Hz, 1H, Ar—H), 7 . . . 74-7.41 (m, 1H, Ar—H), 7.37-7.36 (d, J=16 Hz, 1H, Ar—H), 7.32 (s, 1H), 7.28 (s, 1H), 7.06-7.05 (m, 1H), 7.02-6.98 (m, 1H), 6.95-6.93 (d, J=Hz, 1H), 4.01-4.02 (m, 4H), 2.1-2.17 (m, 2H), 1.86-1.79 (m, 2H), 1.65-1.47 (m, 4H), 1.22 (m, 3H)∘

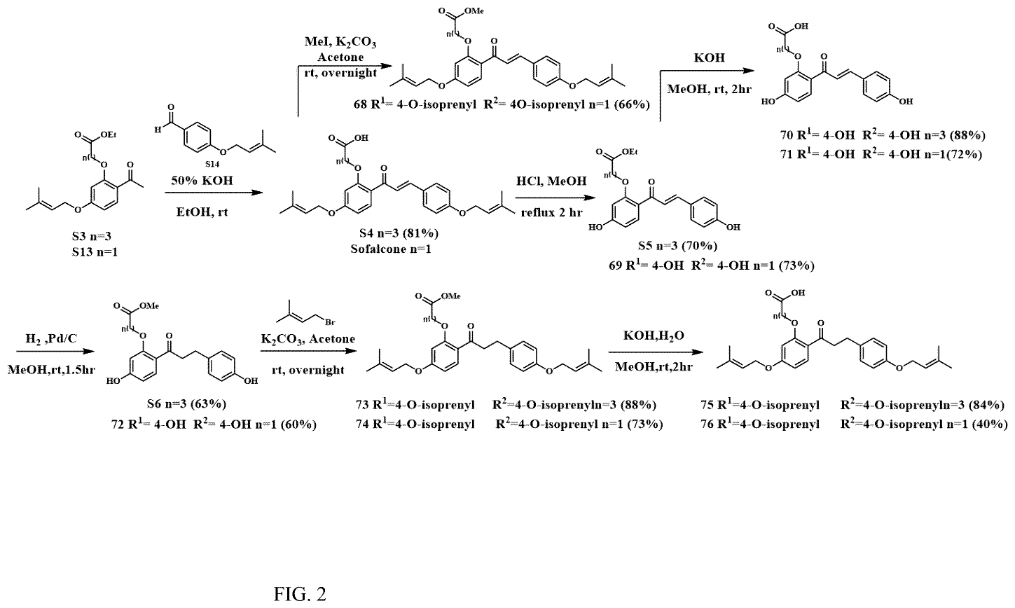

In the compound preparation method shown in FIG. 2, the preparation methods of Compounds 74 and 76 are provided by the present invention as exemplar descriptions.

### Preparation of Compound 74

Step 1: starting compound S13 (0.92 g, 3 mmol) was placed in a reaction bottle, ethanol was used as the solvent, and a magnet was added for stirring. 50% KOH was slowly added dropwise and allowed it to stand for 10 minutes. Then, S14 (0.57 g, 3 mmol) previously dissolved in ethanol was added into the reaction bottle dropwise, and stirred at room temperature for 12-16 hours. TLC sheet was used to confirm that the reaction was complete, then precipitation of solid suspensions could be observed. The solvent was removed with a rotary evaporator, 200 mL of water was added, acidified with 6N HCL until the pH value was equal to 4, and then the crude product was extracted with ethyl acetate (EA). Then, purified with column chromatography by using EA:n-hexane (hex)=1:1 as the eluent. Compound sofalcone could be obtained by using ethanol for recrystallization.

Step 2: sofalcone (1.4 g, 3 mmol) was placed in a reaction bottle and methanol was used as the solvent. After adding a magnet and stirring, 3 ml of concentrated hydrochloric acid was slowly added dropwise, heated to reflux for 1-2 hours. After confirming completion of the reaction with a TLC sheet, the solvent was removed with a rotary evaporator, 200 mL of water was added, and the crude product was extracted with EA. Then, Compound 69 could be obtained by purification with column chromatography.

Step 3: Compound 69 (0.98 g, 3 mmol) was placed in a reaction bottle and a palladium on carbon Pd/C (0.11 g, 1 mmol) was added, the reaction bottle was suctioned to vacuum and methanol was used as the solvent, then hydrogen gas was introduced and reacted at room temperature for 1-3 hours. After monitoring the reaction with a TLC sheet to confirm completion of the reaction, the reaction mixture was filtered through a filter filled with diatomaceous earth under reduced pressure and the solvent was removed with a rotary evaporator. Compound 72 could be obtained after purification with column chromatography.

Step 4: Compound 72 (0.99 g, 3 mmol) and potassium carbonate (2.1 g, 15 mmol) were placed in a reaction bottle and acetone was added as the solvent, after adding a magnet for stirring, isoprenyl bromide (1.88 g, 12 mmol) was slowly added dropwise and reacted at room temperature for 12-16 hours. After monitoring with a TLC sheet to confirm completion of the reaction, the solvent was removed with a rotary evaporator. 200 mL of water was added and neutralized with 6N HCl until the pH value was equal to 7, and the crude product was extracted with EA. Then, Compound 74 could be obtained by purification using EA:n-hexane (hex)=1:5 as the eluent.

¹H NMR (400 MHZ, CDCl₃) δ 7.83-7.81 (d, J=8 Hz, 1H, Ar—H), 7.16-7.14 (d, J=8 Hz, 2H, Ar—H), 6.84-6.82 (d, J=8 Hz 2H, Ar—H), 6.58-6.56 (d, 1H, Ar—H), 5.51-5.44 (m, 2H), 4.66 (s, 2H), 4.54-4.46 (dd, J −32 Hz, 4H), 3.73 (s, 3H), 3.36-3.43 (t, 2H), 2.96 (t, 3H,), 1.81-1.78 (m, 6H), 1.75-1.73 (m, 6H)∘

### Preparation of Compound 76

Compound 74 (1.4 g, 3 mmol) was placed in a reaction bottle, methanol was added as the solvent, and a magnet was added for stirring. 50% KOH was slowly added dropwise and reacted at room temperature for 1-2 hours. After confirming completion of the reaction with a TLC sheet, the solvent was removed with a rotary evaporator, 200 mL of water was added, acidified with 6N HCl until Ph=4, and the crude product was extracted with EA. Then, Compound 76 could be obtained by purification with column chromatography using EA:n-hexane (hex)=1:5 as the eluent.

¹H NMR (400 MHZ, CDCl₃) δ 7.72-7.70 (d, J=8 Hz, 1H, Ar—H), 7.12-7.11 (d, J=4 Hz, 2H, Ar—H), 6.84-6.82 (d, J=16 Hz, 2H, Ar—H), 6.59-6.44 (m, 2H, Ar—H), 5.48-5.44 (m, 2H), 4.69-4.46 (m, 6H), 3.21 (s, 2H), 2.96 (s, 2H), 1.80-1.73 (m, 12H)∘

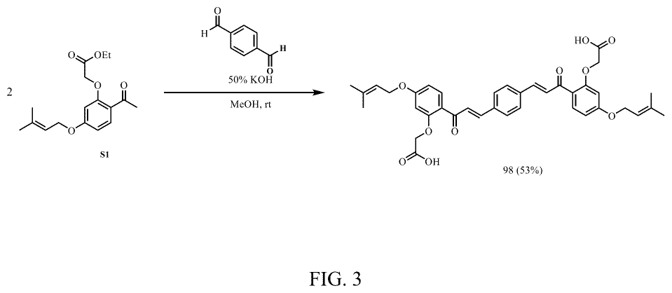

In the compound preparation method shown in FIG. 3, the preparation method of Compound 98 is provided by the present invention as exemplar description.

### Preparation of Compound 98

Starting compound S1 (1.23 g, 4 mmol) was placed in a reaction bottle, ethanol was added as the solvent, and a magnet was added for stirring. 50% KOH was slowly added dropwise and allowed it to stand for 10 minutes. Then terephthalaldehyde (0.27 g, 2 mmol) previously dissolved in ethanol was added dropwise into the reaction bottle, and stirred at room temperature for 12-16 hours. After monitoring with a TLC sheet to confirm completion of the reaction, precipitation of solid suspensions could be observed. The solvent was removed with a rotary evaporator, 200 mL of water was added, acidified with 6N HCl until the pH value was equal to 4, and then the crude product was extracted with ethyl acetate (EA). Then, purified with column chromatography using EA:n-hexane (hex)=1:1 as the eluent. Compound 98 could be obtained by recrystallization with ethanol.

¹H NMR (400 MHZ, DMSO-d6) δ 8.11-8.08 (d, J=12 Hz, 4H, Ar-H), 7.81 (s, 4H, Ar—H), 6.67-6.49 (m, 2H,), 5.45-5.42 (m, 2H), 4.87 (m, 4H), 4.62-4.61 (d, J=4, 4H), 1.75-1.73 (m, 12H), 2.96 (s, 2H)∘

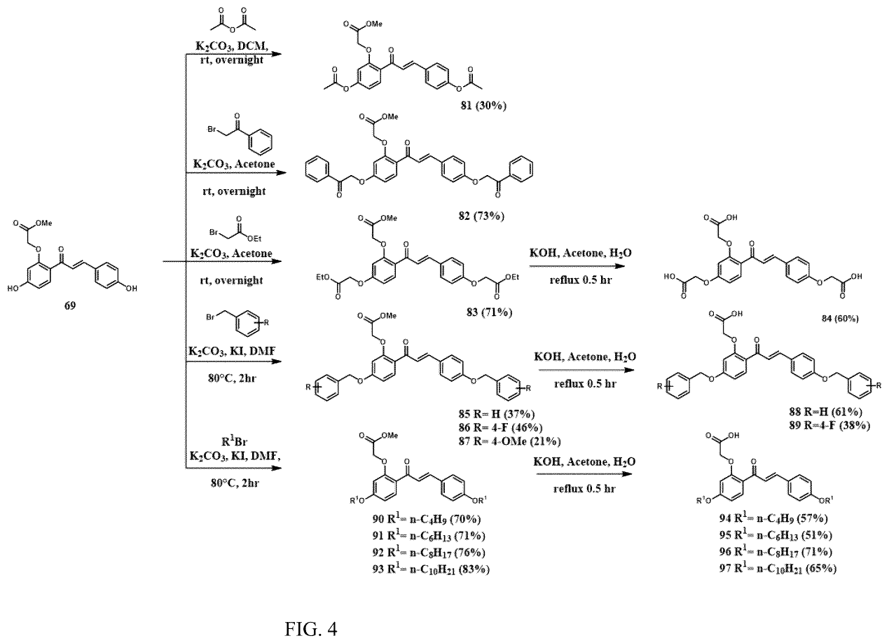

In the compound preparation method shown in FIG. 4, the preparation method of Compounds 83 and 84 are provided by the present invention as exemplar descriptions.

### Preparation of Compound 83

Compound 69 (0.98 g, 3 mmol) and potassium carbonate (2.1 g, 15 mmol) were placed in a reaction bottle with acetone as the solvent, ethyl 2-bromoacetate (1.00 g, 6 mmol) was slowly added dropwise and reacted at room temperature for 12-16 hours. After monitoring with a TLC sheet to confirm completion of the reaction, the solvent was removed with a rotary evaporator, 200 ml of water was added, neutralized with 6N HCl until the pH value was equal to 7, and the crude product was extracted with EA. Then, Compound 83 could be obtained by purification with column chromatography using EA:n-hexane (hex)=1:5 as the eluant.

¹H NMR (400 MHZ, DMSO-d6) δ 7.72-7.59 (m, 6H, Ar—H), 7.00-6.98 (d, J=8 Hz, 2H, Ar—H), 6.71-6.70 (m, 1H, Ar—H), 5.01 (s, 2H), 4.89-4.85 (d, J=16 Hz, 4H), 3.72 (s, 3H), 1.25-1.20 (m, 6H)∘

### Preparation of Compound 84

Compound 83 (1.5 g, 3 mmol) was placed in a reaction bottle, acetone was added as the solvent, and a magnet was added for stirring. 50% KOH was slowly added dropwise and reacted at room temperature for 1-2 hours. After confirming completion of the reaction with a TLC sheet, the solvent was removed with a rotary evaporator, 200 mL of water was added, acidified with 6N HCl until pH-4, and the crude product was extracted with EA. Then, Compound 84 could be obtained by purification with column chromatography using EA:n-hexane (hex)=1:1 as the eluent.

¹H NMR (400 MHZ, DMSO-d6) δ 7.89-7.85 (d, J=16 Hz, 1H, Ar-H), 7.75-7.23 (d, J=8 Hz, 1H, Ar—H), 7.68-7.62 (t, 2H, Ar—H), 7.58 (s, 1H, Ar—H), 6.96-6.94 (d, J=8 Hz, 1H, Ar—H), 6.70-6.69 (d, J=4 Hz, 6H, Ar-H), 6.65-6.63 (dd, J=8 Hz, 1H), 4.90 (s, 2H), 4.79-7.75 (d, J=16 Hz, 4H)∘

### 2. Preparation of Human Platelet Suspension

Blood samples were collected from healthy donors, and acid-citrate-dextrose (ACD) solution was used as an anticoagulant. The blood samples were centrifuged to obtain platelets, and residual plasma was removed by washing to prepare platelet suspension.

### 3. Platelet Aggregation Reaction Test

In the experimental method, a platelet aggregometer (Chrono-Log Co., Havertown, PA, USA) was used for tests. The compound to be tested was added to the platelet suspension and reacted for 3 minutes at 37° C. with stirring (1200 rpm). Then, platelet stimulator U46619 or collagen was added to initiate platelet aggregation, and the test time was 5 minutes. The principle of the platelet aggregation test was: when platelets were stimulated to induce aggregation reaction, the light transmission of the platelet suspension would increase; by calculating changes in light transmission of the platelet suspension before and after being stimulated, the percentage of platelet aggregation could be obtained.

### 4. Measurement of Platelet TxB2 and PGE2 Levels

The compound to be tested was added to the platelet suspension and reacted for 3 minutes at 37° C. under stirring (1200 rpm), then arachidonic acid (AA) (100 μM) was added to react for 4 minutes, and finally EDTA (5 mM) was added to terminate the reaction. The above sample was centrifuged at 13,000 rpm for 1 minute at 4° C. and the supernatant was collected. The concentrations of TxB2 and PGE2 in the supernatant were measured respectively using enzyme immunoassay kits (TxB2 ELISA kit and PGE2 ELISA kit; Cayman Chemical Company).

### 5. COX1 and COX2 Enzyme Activity Assays

COX1 and COX2 enzyme activities were analyzed by using a human COX inhibitor screening assay kit (Cayman Chemical Company). After allowing the to-be-tested compound, human recombinant COX1 or COX2 enzyme and heme to stand at 37° C. for 15 minutes, AA (10 μM) was added to react for 2 minutes. Then, stannous chloride was added to reduce PGH2, the product of the above COX reaction, to PGF2α. Finally, the PGF2α level in the sample was measured by using enzyme immunoassay, and the inhibition percentage was calculated.

### 6. Test of Ferric Chloride-Induced Carotid Artery Thrombosis in Mice

In this experiment, a ferric chloride-induced arterial injury was used to evaluate the antithrombotic effect of the to-be-tested compound. The to-be-tested compound was orally administered by gavage to BALB/c mice aged approximately 6-8 weeks. Then, the mice were anesthetized with isoflurane, the right carotid artery was exposed through surgery, and filter paper (2×4 mm²) containing 8.5% ferric chloride was attached to the right carotid artery for 3 minutes. After removing the filter paper, remaining ferric chloride was wiped off with cotton balls soaked with physiological saline, and an ultrasonic imaging system (VEVO 2100 system, VisualSonics) equipped with a Doppler flow probe (MS400, 18-38 MHz) was used to monitor the blood flow of the right carotid artery. The occlusion time was determined when the blood flow completely stopped for at least 1 minute. If the occlusion time was longer than 30 minutes, it was considered the end point of the experiment.

### 7. Mouse Bleeding Test

In the present invention, the effect of the to-be-tested compound on normal hemostasis was evaluated by docking off the tail ends of the mice to cause bleeding. The to-be-tested compound was orally administered to BALB/c mice aged approximately 6-8 weeks by gavage. Then, the mice were anesthetized with isoflurane, and 2 mm of the distal end of their tails were excised. The tails of the mice were immersed in physiological saline at 37° C., and the bleeding time was observed. The determination of bleeding time was: the time required from the time the mouse tail was cut off until the bleeding completely stopped. If the bleeding did not stop after 15 minutes, it was considered the end point of the experiment.

### 8. Statistical Analysis

The experimental results of the present invention are expressed as mean±standard error of the mean. Statistical significance was calculated by using the GraphPad Prism software, one-way analysis of variance (One-way ANOVA); when the p value <0.05, it was considered statistically significant.

### Results

### 1. Anti-Platelet Aggregation Effect of Sofalcone Derivatives

The present invention divided the sofalcone derivatives prepared according to FIG. 1 to FIG. 4 into two categories: (A) the compounds of the first category included the compounds having formula (I):

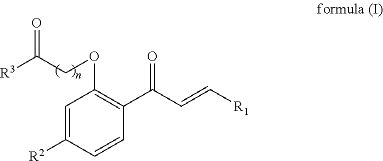

wherein the compounds having formula (I) comprised derivatives 1-48, 51-71, 77-99, and 101-114; and (B) the compounds of the second category included the compounds having formula (II):

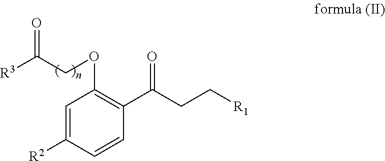

wherein the compounds having formula (II) comprised derivatives 49, 50, 72-76, 100, 115, and 116.

Table 1 shows that sofalcone (SFC) derivatives that could effectively inhibit platelet aggregation caused by TxA2 agonist U46619 or collagen, and their potency was better than clinical drugs such as aspirin and seratrodast.

TABLE 1
Chemical structures and antiplatelet activities of sofalcone derivatives

| | | | | | U46619 | Collagen |
|---|---|---|---|---|---|---|
| | | | | | IC₅₀ | IC ₅₀ |
| Compound | R¹ | R² | R³ | n | (μM) | (μM) |
| SFC | Ph-4-O-isoprenyl | O-isoprenyl | OH | 1 | 15.98 | 48.96 |
| Aspirin | — | — | — | — | >200 | 78.95 |
| Seratrodast | — | — | — | — | 0.71 | 5.27 |
| 1 | Ph-2-Fluoro | H | OH | 5 | 0.51 | 0.07 |
| 2 | Ph-3-Fluoro | H | OH | 5 | 0.05 | 0.05 |
| 3 | Ph-4-Fluoro | H | OH | 3 | 0.07 | 0.03 |
| 4 | Ph-4-O-geranyl | O-geranyl | OH | 3 | 0.05 | 0.06 |
| 5 | Ph-4-O-isoprenyl | O-geranyl | OH | 3 | 4.45 | 6.28 |
| 6 | Ph-4-O-geranyl | O-geranyl | OH | 3 | 3.66 | 44.36 |
| 7 | Ph | O-geranyl | OH | 3 | 0.49 | 4.03 |
| 8 | Ph-4-O-geranyl | H | OEt | 3 | 2.78 | 4.24 |
| 9 | Ph-4-O-isoprenyl | Fluoro | OH | 3 | 5.44 | 5.14 |
| 10 | Ph-4-O-geranyl | Fluoro | OH | 3 | 5.24 | 52.47 |
| 11 | Ph | Fluoro | OH | 5 | 54.39 | 5.38 |
| 12 | Ph-4-Fluoro | Fluoro | OH | 5 | 0.50 | 0.43 |
| 13 | Ph-2-Fluoro | O-isoprenyl | OH | 3 | 0.51 | 0.65 |
| 14 | Ph-3-Fluoro | O-isoprenyl | OH | 3 | 5.18 | 7.05 |
| 15 | Ph-4-Fluoro | O-isoprenyl | OH | 3 | 0.05 | 0.35 |
| 16 | Ph-4-Fluoro | Fluoro | OH | 5 | 0.37 | 0.62 |
| 17 | Ph-4-O-isoprenyl | O-isoprenyl | OH (potassium salt) | 3 | 0.04 | 0.32 |
| 18 | Ph-4-OMe | OMe | OH | 1 | >100 | 96.22 |
| 19 | Ph-2-O-isoprenyl | O-isoprenyl | OH | 1 | 20.34 | 15.25 |
| 20 | Ph-3-O-isoprenyl | O-isoprenyl | OH | 1 | 20.60 | 28.30 |
| 21 | Ph-3,4- O-isoprenyl | O-isoprenyl | OH | 1 | 14.39 | 29.69 |
| 22 | Ph-4-H | O-isoprenyl | OH | 1 | 43.18 | 12.78 |
| 23 | Ph-4-Fluoro | O-isoprenyl | OH | 1 | 22.23 | 33.54 |
| 24 | Ph-4-Chloro | O-isoprenyl | OH | 1 | 43.42 | 67.59 |
| 25 | Ph-4-NO₂ | O-isoprenyl | OH | 1 | 43.76 | 49.21 |
| 26 | Ph-4-NHAc | O-isoprenyl | OH | 1 | 15.69 | 40.78 |
| 27 | Ph-4-O-isoprenyl | H | OH | 1 | 5.07 | 18.83 |
| 28 | Ph-4-Fluoro | H | OH | 1 | 1.80 | 4.22 |
| 29 | Ph | Fluoro | OH | 1 | 1.56 | 7.88 |
| 30 | Ph-4-O-isoprenyl | O-isoprenyl | OH | 3 | 0.04 | 0.38 |
| 31 | Ph | Fluoro | OH | 1 | 4.4 | 7.1 |
| 32 | Ph-4-O-isoprenyl | O-isoprenyl | OH | 5 | 5.85 | 12.84 |
| 33 | Ph-4-Fluoro | Fluoro | OH | 1 | 2.15 | 12.18 |
| 34 | Ph | H | OH | 3 | 9.5 | 6.4 |
| 35 | Ph | H | OH | 1 | 6.58 | 7.91 |
| 36 | Ph | H | OH | 5 | 0.19 | 0.13 |
| 37 | Ph-4-O-isoprenyl | O-isoprenyl | OH | 4 | 0.23 | 1.58 |
| 38 | Ph-2-Fluoro | H | OEt | 5 | 0.03 | 0.07 |
| 39 | Ph-3-Fluoro | H | OEt | 5 | 0.05 | 0.07 |
| 40 | Ph-3-OH | OH | OEt | 1 | 25.90 | 10.32 |
| 41 | Ph-3,4-OH | OH | OEt | 1 | 34.92 | 15.17 |
| 42 | Ph-4-OH | H | OEt | 1 | 38.67 | 1.59 |
| 43 | Ph-4-Fluoro | OH | OEt | 1 | 55.62 | 2.26 |
| 44 | Ph-4-Chloro | OH | OEt | 1 | >100 | 8.92 |
| 45 | Ph-4-NO₂ | OH | OEt | 1 | >20 | >20 |
| 46 | Ph-4-NH₂ | H | OEt | 1 | 64.01 | 5.08 |
| 47 | Ph-4-OH | H | OEt | 1 | 35.2 | 16.36 |
| 48 | Ph-4-OMe | OMe | OEt | 1 | >100 | >100 |
| 49 | Ph | H | OH | 5 | 0.5 | 4.85 |
| 50 | Ph-3-Fluoro | H | OH | 5 | 0.57 | 0.33 |
| 51 | 2-thiophenyl | H | OH | 5 | 0.57 | 0.62 |
| 52 | 2-furanyl | H | OH | 5 | 0.50 | 0.56 |
| 53 | 2-pyridinyl | H | OH | 5 | 50.4 | 44.44 |
| 54 | 3-pyridinyl | H | OH | 5 | 5.45 | 22.14 |
| 55 | 2-thiophenyl | O-isoprenyl | OH | 5 | 5.37 | 4.40 |
| 56 | 2-furanyl | H | OH | 1 | 72.19 | 38.27 |
| 57 | 2-thiophenyl | H | OH | 1 | 71.51 | 15.46 |
| 58 | 2-(5-Bromothiophenyl) | H | OH | 1 | 72.58 | 40.88 |
| 59 | 2-pyridinyl | H | OH | 1 | 72.06 | 33.79 |
| 60 | 3-pyridinyl | H | OH | 1 | 29.30 | 39.77 |
| 61 | 2-thiophenyl | H | OEt | 5 | 0.54 | 0.56 |
| 62 | 2-furanyl | H | OEt | 5 | 0.48 | 0.58 |
| 63 | 2-pyridinyl | H | OEt | 5 | 4.57 | 6.05 |
| 64 | 2-thiophenyl | O-isoprenyl | OEt | 3 | 5.87 | 3.85 |
| 65 | 2-furanyl | H | OEt | 1 | 78.41 | 3.13 |
| 66 | 2-thiophenyl | H | OEt | 1 | 70.18 | 2.86 |
| 67 | 2-(5-Bromothiophenyl) | H | OEt | 1 | >100 | 5.35 |
| 68 | Ph-4-O-isoprenyl | O-isoprenyl | OMe | 1 | >100 | >100 |
| 69 | Ph-4-OH | OH | OMe | 1 | 27.82 | 3.42 |
| 70 | Ph-4-OH | OH | OH | 3 | 5.43 | 6.55 |
| 71 | Ph-4-OH | OH | OH | 1 | 65.41 | >100 |
| 72 | Ph-4-OH | OH | OMe | 1 | 47.63 | 11.60 |
| 73 | Ph-4-O-isoprenyl | O-isoprenyl | OMe | 3 | 10.7 | 62.87 |
| 74 | Ph-4-O-isoprenyl | O-isoprenyl | OMe | 1 | >100 | >100 |
| 75 | Ph-4-O-isoprenyl | O-isoprenyl | OH | 3 | 0.35 | 1.3 |
| 76 | Ph-4-O-isoprenyl | O-isoprenyl | OH | 1 | 12.57 | 31.02 |
| 77 | Ph | H | OH | 5 | 5.54 | 43.59 |
| 78 | 2-(3-aminothiophenyl) | H | OH | 5 | 5.87 | 7.70 |
| 79 | 2-(4-methyl-2-amino- | H | OH | 5 | 0.57 | 0.66 |
| | thiophenyl) | | | | | |
| 80 | 2-(1,3-thiazolyl) | H | OH | 5 | 51.52 | 62.66 |
| 81 | Ph-4-OAc | OAc | OMe | 1 | 28.04 | 23.84 |
| 82 | Ph-4-OCH₂COPh | OCH₂COPh | OMe | 1 | >100 | 100 |
| 83 | Ph-4-OCH₃COOEt | OCH₃COOEt | OMe | 1 | 8.62 | 8.31 |
| 84 | Ph-4-OCH₃COOEt | OCH₃COOEt | OH | 1 | 80.84 | >100 |
| 85 | Ph-4-OBn | 4-OBn | OMe | 1 | >100 | >100 |
| 86 | Ph-4-O-(4-Fluorobenzyl) | 4-O-(4-Fluorobenzyl) | OMe | 1 | >100 | >100 |
| 87 | Ph-4-O-(4-Methoxylbenzyl) | 4-O-(4-Methoxylbenzyl) | OMe | 1 | >100 | >100 |
| 88 | Ph-4-OBn | 4-OBn | OH | 1 | 9.09 | 45.07 |
| 89 | Ph-4-O-(p-Fluorobenzyl) | 4-O-(p-Fluorobenzyl) | OH | 1 | 3.59 | 33.93 |
| 90 | Ph-4-O(CH₂)₃CH₃ | 4-O(CH₂)₃CH₃ | OMe | 1 | >100 | >100 |
| 91 | Ph-4-O(CH₂)₅CH₃ | 4-O(CH₂)₅CH₃ | OMe | 1 | >50 | >50 |
| 92 | Ph-4-O(CH₂)₇CH₃ | 4-O(CH₂)₇CH₃ | OMe | 1 | >50 | >50 |
| 93 | Ph-4-O(CH₂)₉CH₃ | 4-O(CH₂)₉CH₃ | OMe | 1 | >50 | >50 |
| 94 | Ph-4-O(CH₂)₃CH₃ | 4-O(CH₂)₃CH₃ | OH | 1 | 40.69 | 63.25 |
| 95 | Ph-4-O(CH₂)₅CH₃ | 4-O(CH₂)₅CH₃ | OH | 1 | 36.92 | 76.29 |
| 96 | Ph-4-O(CH₂)₇CH₃ | 4-O(CH₂)₇CH₃ | OH | 1 | >100 | >100 |
| 97 | Ph-4-O(CH₂)₉CH₃ | 4-O(CH₂)₉CH₃ | OH | 1 | >50 | >50 |
| 98 | — | — | OH | 2 | >50 | >50 |
| 99 | Ph-4-O-isoprenyl | H | OH | 3 | 3.33 | 6.42 |
| 100 | Ph-4-O-ipentyl | 4-O-ipentyl | OH | 1 | 1.73 | 66.79 |
| 101 | Ph | H | OH | 4 | 3.53 | 3.25 |
| 102 | Ph-4-O-isoprenyl | H | OH | 4 | 2.42 | 3.14 |
| 103 | Ph-4-O-isoprenyl | H | OH | 5 | 0.46 | 0.38 |
| 104 | Ph | H | OEt | 6 | 13.48 | 5.31 |
| 105 | Ph | H | OH | 6 | 2.92 | 9.08 |
| 106 | Ph | H | OEt | 7 | 2.84 | 13.07 |
| 107 | Ph | H | OH | 7 | 2.49 | 8.35 |
| 108 | Ph | H | OEt | 1 | 15.33 | 3.67 |
| 109 | Ph | H | OEt | 3 | 9.56 | 4.89 |
| 110 | Ph | H | OEt | 4 | 2.90 | 4.48 |
| 111 | Ph | H | OEt | 5 | 0.16 | 0.22 |
| 112 | Ph | H | OMe | 3 | 13.41 | 2.69 |
| 113 | Ph | H | OMe | 5 | 0.57 | 0.35 |
| 114 | Ph | H | OMe | 5 | 0.10 | 0.09 |
| 115 | Ph | H | OEt | 5 | 0.2 | 5.87 |
| 116 | Ph | H | OH | 5 | 0.2 | 4.46 |

2. Platelet Aggregation Induced by Competitively Antagonizing U46619 with Sofalcone and its Derivatives

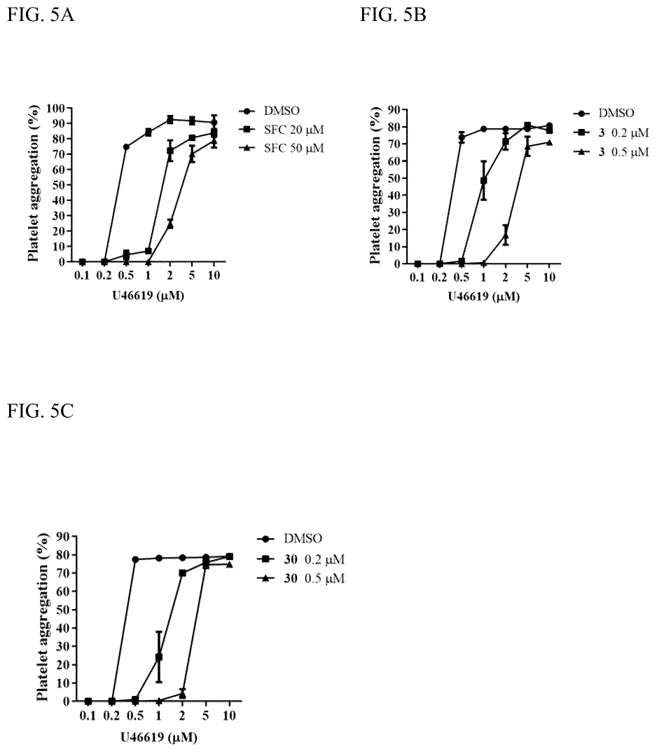

FIG. 5A to FIG. 5C show that sofalcone and its derivatives 3 and 30 could inhibit platelet aggregation induced by U46619 (100 μM) in human platelet suspensions. After pretreating human platelet suspensions with dimethylsulfoxide (DMSO), sofalcone (20 μM and 50 μM), and sofalcone derivatives 3 or 30 (0.2 μM and 0.5 μM, respectively), different concentrations of U46619 (0.1-10 μM) were added to stimulate platelet aggregation. In the presence of sofalcone and its derivatives, the U46619 concentration-platelet aggregation response curve shifted parallelly to the right, but the maximum level of platelet aggregation was not significantly affected. According to the above results, it indicated that sofalcone and its derivatives 3 or 30 could competitively antagonize TxA2 receptor.

### 3. The Effect of Sofalcone and its Derivatives on Human Platelet AA Metabolites

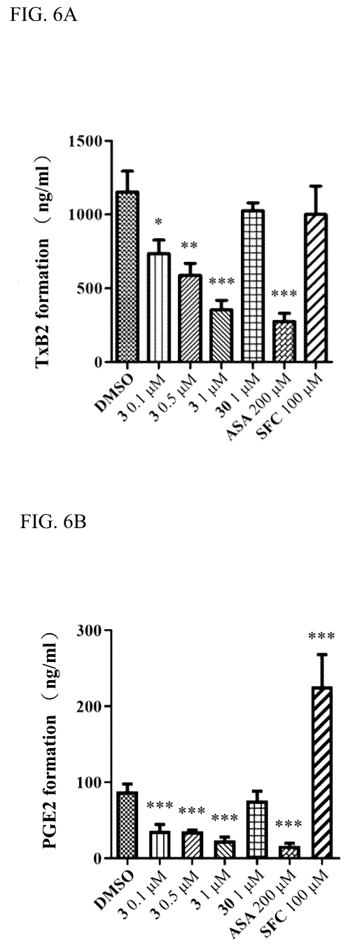

FIG. 6A and FIG. 6B show the effect of sofalcone and its derivatives 3 and 30 on the formation of TxB2 (a stable metabolite of TxA2) and PGE2 induced by arachidonic acid (AA) (100 μM) in human platelet suspensions. Sofalcone and its derivative 30 did not inhibit the formation of platelet TxB2 and PGE2. However, similar to aspirin, derivative 3 also inhibited the formation of TxB2 and PGE2. According to the above results, it indicated that in addition to TxA2 receptor antagonism, derivative 3 could also reduce the formation of TxA2.

### 4. The Effects of the Sofalcone Derivatives on Human Recombinant COX1 and COX2

Table 2 shows that derivative 3 inhibited the enzyme activities of human recombinant COX1 and COX2 in a dose-related manner, and had a better inhibitory effect on COX1. According to the above results, it indicated that derivative 3 could reduce the synthesis of platelet TxA2 by inhibiting COX1.

TABLE 2
The inhibitory effect of derivative
3 on human recombinant COX1 and COX2

| | COX1 | COX2 |
|---|---|---|
| | (Inhibition rate %) | (Inhibition rate %) |
| 3 1 μM | 19.1 | N.D. |
| 3 10 μM | 61.6 | 26.7 |
| 3 100 μM | 89.8 | 48.6 |
| Aspirin 200 μM | 26.0 | N.D. |
| Celecoxib 1 μM | N.D. | 66.3 |

N.D.: No significant difference

### 5. Antithrombotic Effect of Sofalcone and its Derivatives in Animal Models

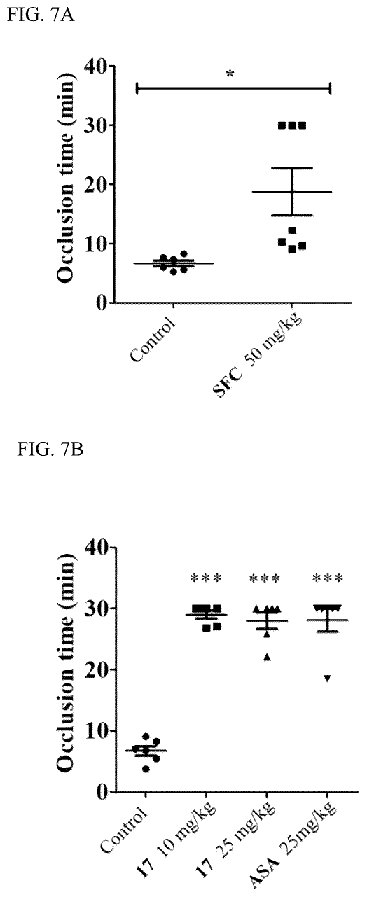

FIG. 7A and FIG. 7B show that sofalcone (at a dose of 50 mg/kg per day for 4 days) or derivative 17 (potassium salt of derivative 30) (single dose only, 10 or 25 mg/kg) was orally administered to mice by gavage. In the FeCl₃-induced carotid artery thrombosis mode, the carotid artery occlusion time was significantly prolonged, confirming its in vivo anti-thrombotic effect. In comparison, aspirin (ASA) (25 mg/kg) also prolonged the carotid artery occlusion time.

### 6. The Effect of Sofalcone and its Derivatives on Bleeding Time in Animal Models

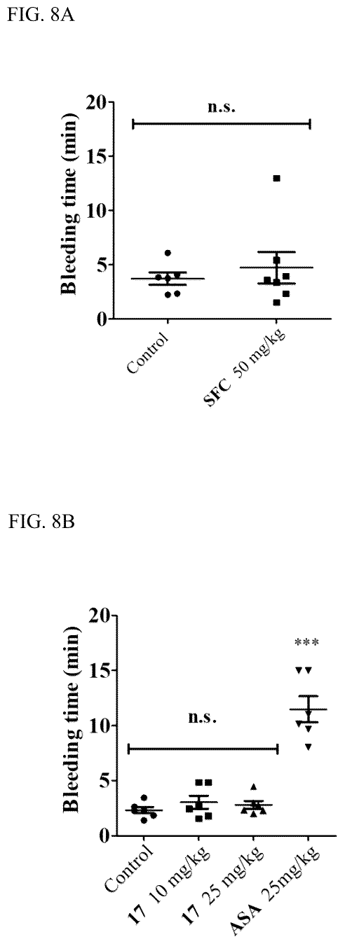

FIG. 8A and FIG. 8B show that when sofalcone (at a dose of 50 mg/kg per day for 4 days) or derivative 17 (potassium salt of 30) (only a single dose of 10 or 25 mg/kg) was administered to mice by gavage, it did not affect the tail bleeding time of the mice, confirming that it did not inhibit normal hemostatic function. In comparison, aspirin (ASA) (25 mg/kg) significantly prolonged the tail bleeding time of the mice.

The present invention is appropriately described so that it may be practiced with elements or limitations not specifically disclosed herein. The terms that are used to describe are not limiting. No distinction is made between the expressions and descriptions using these terms and any equivalents thereto, but it should be recognized that the rights within the present invention may be modified. Therefore, although the present invention is described in terms of embodiments and other aspects, the contents disclosed herein may be modified and varied by those skilled in the art, and such modifications and variations are deemed to be within the scope of the present invention.

What is claimed is:

1. A compound or a pharmaceutically acceptable salt thereof, wherein the compound has a structure of formula (I): 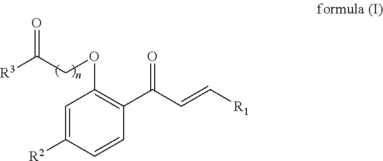
wherein R¹ is none, H, phenyl, thiophenyl, furanyl, pyridinyl, bromothiophenyl, thiazolyl, or X-substituted phenyl, and X is H, halogen, NO₂, NH₂, NHAc, O-Ac, O-geranyl, C₁₋₆ alkyl, O—C₁₋₁₀ alkyl, OH, OBn, aminothio, O-isoprenyl, O-halobenzyl, O—C₁₋₆ alkoxybenzyl, O—C₁₋₆ alkyl-CO-phenyl, or O—C₁₋₆ alkyl-COO—C₁₋₆ alkyl;
R² is none, H, OH, halogen, O-geranyl, O-isoprenyl, O—C₁₋₁₀ alkyl, O-Ac, OBn, O-halobenzyl, O—C₁₋₆ alkyloxybenzyl, phenyl-O-isoprenyl, O—C₁₋₆ alkyl-CO-phenyl, or O—C₁₋₆ alkyl-COO—C₁₋₆ alkyl;
R³ is OH or O—C₁₋₆ alkyl; and
n is 1-7.

2. The compound or the pharmaceutically acceptable salt thereof of claim 1, wherein the R¹ is phenyl-4-fluoro, the R² is H, the R³ is OH, and the n is 5.

3. The compound or the pharmaceutically acceptable salt thereof of claim 1, wherein the R¹ is phenyl-4-O-isoprenyl, the R² is O-isoprenyl, the R³ is OH, and the n is 3-7.

4. The compound or the pharmaceutically acceptable salt thereof of claim 1, wherein the R¹ is phenyl-4-O-isoprenyl group, the R² is O-isoprenyl, and the R³ is OH, and the n is 3.

5. A method for preventing or treating a disease related to platelet aggregation, which comprises administering a composition comprising the compound or the pharmaceutically acceptable salt thereof of claim 1 to a subject suffering from the disease related to platelet aggregation.

6. The method of claim 5, wherein the disease related to platelet aggregation comprises unstable angina, acute coronary syndrome, myocardial infarction, transient ischemic attack, cerebral stroke, atherosclerosis, peripheral occlusive arterial disease, venous thrombosis, thrombophlebitis, arterial embolism, kidney embolism, pulmonary embolism, or thrombotic diseases resulting from medical implants, devices, or procedures in which blood is exposed to artificial surfaces that facilitate thrombosis.

7. A compound or a pharmaceutically acceptable salt thereof, wherein the compound has a structure of formula (II): 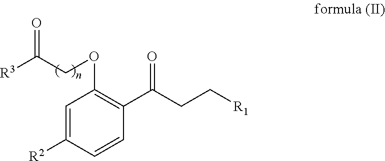
wherein R¹ is phenyl, or Y-substituted phenyl, and Y is halogen, OH, O-isoprenyl, or O—C₁₋₆ alkyl;
R² is H, OH, O-isoprenyl, or O—C₁₋₆ alkyl;
R³ is OH, O—C₁₋₆ alkyl; and
n is 1-7.

8. A method for preventing or treating a disease related to platelet aggregation, which comprises administering a composition comprising the compound or the pharmaceutically acceptable salt thereof of claim 7 to a subject suffering from the disease related to platelet aggregation.

9. The method of claim 8, wherein the disease related to platelet aggregation comprises unstable angina, acute coronary syndrome, myocardial infarction, transient ischemic attack, cerebral stroke, atherosclerosis, peripheral occlusive arterial disease, venous thrombosis, thrombophlebitis, arterial embolism, kidney embolism, pulmonary embolism, or thrombotic diseases resulting from medical implants, devices, or procedures in which blood is exposed to artificial surfaces that facilitate thrombosis.
